(19) United States
(12) Patent Application Publication
(10) Pub. No.: US 2003/0018305 A1
(43) Pub. Date: Jan. 23, 2003

(54) Needleless vascular infusion port apparatus

(76) Inventor: Ming-Hsiung Tsai, Taichung (TW)

Correspondence Address: Ming-Hsiung Tsai, P.O. BOX 2103, Taichung, TW

(21) Appl. No.: 10/194,267

(22) Filed: Jul. 15, 2002

Related U.S. Application Data
(63) Continuation-in-part of application No. 10/162,476, filed on Jun. 5, 2002.

(30) Foreign Application Priority Data
Jul. 17, 2001 (TW) 90212015

(51) Int. Cl. A61M 5/00

(52) U.S. Cl. 604/247

(57) ABSTRACT

Needleless vascular infusion port apparatuses are disclosed. The infusion ports include a needleless valve that permits infusion of a liquid into the infusion port without use of a sharp.

### BACKGROUND

Intravenous infusion systems are common and known in the medical field, so a general background description of them is not provided here. Existing intravenous infusion systems are problematic in that they typically provide an infusion port that can only be accessed by use of a sharp, such as a needle, to infuse a desired infusate fluid, such as medicine, blood, nutrients, electrolyte solution, anesthesia, muscle relaxants, or other desired infusates. For health, safety and convenience reasons, medical personnel often wish to avoid use of a sharp. Use of a sharp involves a risk of inadvertent self-impalement or the impalement of others, introducing risks of injury and disease for both the user of the sharp and for all others present.

### SUMMARY

Needleless and springless vascular infusion port apparatuses are disclosed. The infusion ports include needleless and springless valves that permit infusion of a liquid into the infusion port without use of a sharp, and otherwise maintain a fluid-tight seal at the port location.

### BRIEF DESCRIPTION OF THE DRAWINGS

FIG. 1 depicts an example vascular infusion assembly, including a needleless vascular infusion port.

FIG. 2 depicts a cross sectional side view of an example needleless and springless vascular infusion port apparatus.

FIG. 3 depicts a cross sectional side view of another example needleless and springless vascular infusion port apparatus.

FIG. 4 depicts a top view of an example valve of a needleless and springless vascular infusion port apparatus.

FIG. 5 depicts a top view of another example valve of a needleless and springless vascular infusion port apparatus.

FIG. 6 depicts a cross sectional side view of an example mounting of a needleless and springless valve to an infusion access entry orifice.

FIG. 7 depicts a cross sectional side view of another example mounting of a needleless and springless valve to an infusion access entry orifice.

FIG. 8 depicts a cross sectional side view of another example mounting of a needleless and springless valve to an infusion access entry orifice.

FIG. 9 depicts a side view of an example needleless and springless valve.

FIG. 10 depicts a cross sectional side view of insertion of a non-sharp syringe tip into an example needleless and springless vascular infusion port apparatus.

FIG. 11 depicts examples of locations on a vascular infusion assembly where a needless and springless vascular infusion port may be installed.

### DETAILED DESCRIPTION

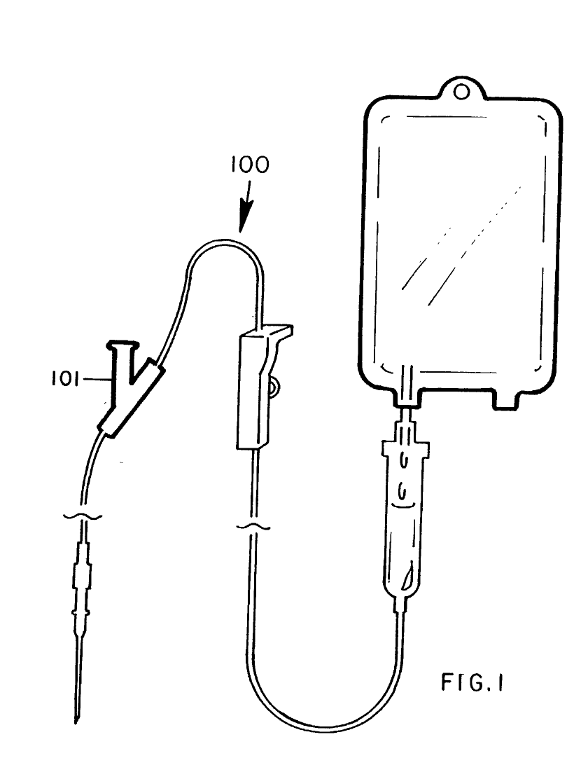

Referring to FIG. 1, an example vascular infusion assembly 100 is depicted. Its various components are known, so a discussion of them is not repeated here. A needleless vascular infusion port 101 is depicted and described in greater detail in light of several examples below.

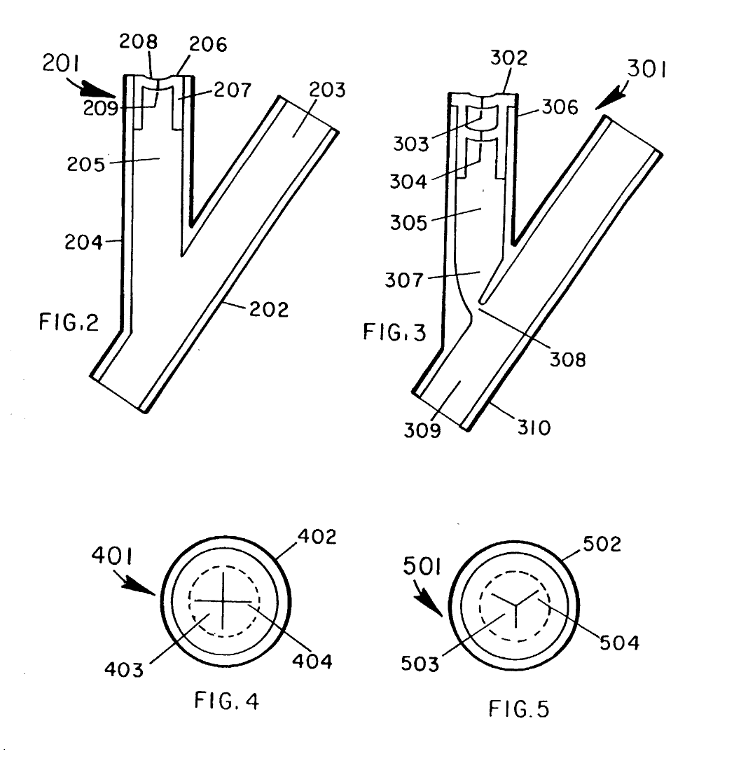

Referring to FIG. 2, a cross sectional side view of an example needleless and springless vascular infusion port apparatus 201 is provided. The apparatus 201 includes a main infusion conduit body 202 through which an internal passageway 203 runs. The infusion conduit body and passageway may be formed as a tube or other geometry as desired. An infusion access 204 is provided projecting from the infusion conduit body 202. The depicted projection is obliquely oriented with respect to the body 202, but could be of any other desired arrangement. The infusion access may be integral with the infusion conduit body or a separate component assembled with it. The infusion access 204 includes an internal passageway 205 that is in fluid communication with the main infusion conduit body internal passageway 203. The internal passageway 205 has a needleless and springless valve assembly 206 installed at its entry orifice. The valve assembly 206 includes a valve body 207, and a valve closure 208 to interrupt the internal passageway 205 and form a fluid-tight seal therein. The valve closure 208 includes a resilient openable diaphragm that has one or more sealable interface surfaces 209, leaves or lips. The diaphragm lips can be parted to form an opening to permit intrusion of a syringe tip therein. Upon removal of the syringe tip, the resiliency of the diaphragm material will cause the diaphragm lips to move back to their original position to re-form and continue to achieve and maintain a fluid-tight engagement and seal for the infusion port. The opening of the diaphragm is achieved by the pliable nature of the diaphragm material. The closing and sealing of the diaphragm is achieved due to the resilient nature of the material, and occurs without the assistance of a mechanical spring. As depicted, the diaphragm is concave when viewed from the distal surface. The concave shape tends to enhance the fluid tightness of the valve in that it increases its resistance to fluid backflow from the proximal side of the valve. The concave shape also tends to cause the tip of a syringe placed against the valve to center on the valve and to be easier to insert through the valve to infuse a liquid therein without use of a sharp.

FIG. 3 depicts a cross sectional side view of another example needleless and springless vascular infusion port apparatus 301. The structures of the needless vascular infusion port apparatus 301 are as described above, with a few exceptions. This apparatus 301 includes a dual diaphragm. The infusion ports include a needleless valve that permits infusion of a liquid into the infusion port without use of a sharp. Valve 302 includes two separate diaphragms 303 and 304 in an in-line or sequential relationship in the internal passageway 305 of the infusion access 306. Use of two diaphragms in sequences may provide a superior fluid-tight seal. The example of FIG. 3 also includes a staging area 307 proximal to the diaphragms and a constrained neck or exit orifice 308 of the infusion access 306. The staging area can receive a quantity of infusate from a syringe whose tip has been inserted through openings in the diaphragms. The constrained neck or exit orifice can be of a diameter that tends to regulate flow of an infusate into the internal passageway 309 of the infusion conduit body 310.

Referring to FIG. 4, a top view of an example valve 401 of a needleless and springless vascular infusion port apparatus. The valve 401 includes an outer body 402, an openable diaphragm portion 403 and a plurality of diaphragm interface surfaces 404 or lips. The outer body sealably interfaces with the internal passageway of an infusion access. That interface may be achieved by a pressure fit, a friction fit, an adhesive, welding, or mechanical fixation. The interface surfaces 404 are in sealed engagement with each other during an ordinary resting state. When a syringe tip absent a needle or sharp is forced through the diaphragm 403 by pushing an opening between the lips by exertion of a parting force, an opening is formed in the valve permitting infusion of a liquid therethrough. When the syringe tip is withdrawn from the valve, the lips will tend to return to their original sealed position due to a resilience and shape memory of the material of the valve. The interface surfaces arrangement depicted in FIG. 4 is referred to as a bifoliate, cross or ‘X’ configuration.

FIG. 5 depicts a top view of another example valve 501 of a needleless and springless vascular infusion port apparatus. The valve 501 includes an outer body 502, an openable diaphragm portion 503 and a plurality of diaphragm interface surfaces 504 or lips. The interface surfaces arrangement depicted in FIG. 5 is referred to as a trifoliate configuration. In addition to the two interface surfaces arrangements depicted in FIGS. 4 and 5, a single slit, multiple slits or leaves, a flap, a trap door, or other configurations could be used to present a valve with diaphragm interfaces that permit protrusion of a non-sharp syringe tip therethrough to infuse a liquid into a vascular infusion assembly.

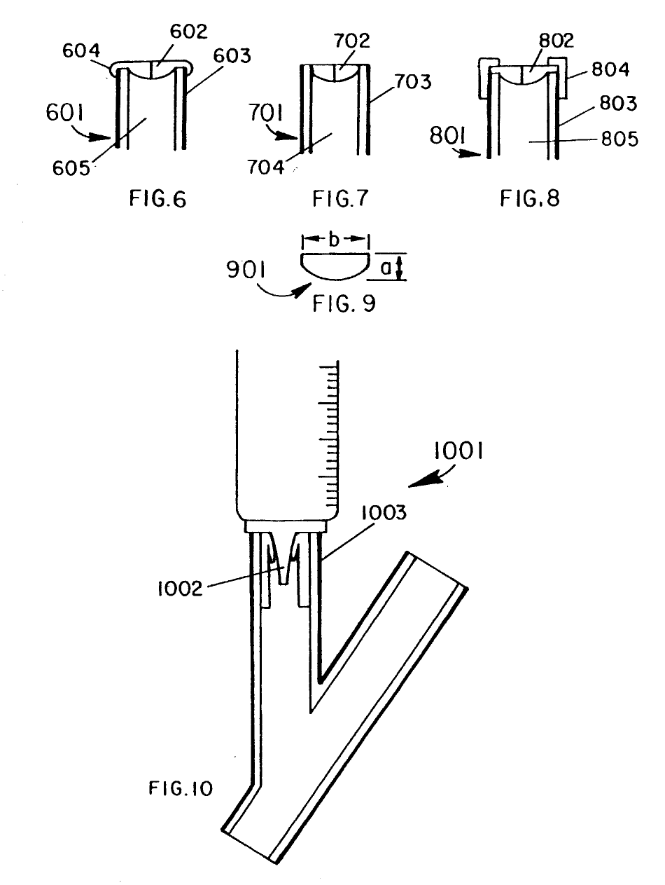

FIG. 6 depicts a cross sectional side view 601 of an example mounting of a needleless and springless valve to an infusion access entry orifice. The view depicts an infusion access 603 and a valve 602 generally as previously described. The valve 602 in this instance is mounted in a wrap around fashion so that the valve subsists within and sealably blocks the infusion access 603 internal passageway 605. The wrap around mounting is defined by a rim or lip 604 of material of the valve body being located about the exterior circumference of the infusion access 603 in order to permanently mount the valve to the infusion access.

FIG. 7 depicts a cross sectional side view 701 of another example mounting of a needleless and springless valve to an infusion access entry orifice. The view depicts an infusion access 703 and a valve 702 generally as previously described. The valve 702 in this instance is mounted completely within the interior of the infusion access 703 in order to sealably block the infusion access 703 internal passageway 704 and to permanently retain the valve therein.

FIG. 8 depicts a cross sectional side view 801 of another example mounting of a needleless and springless valve to an infusion access entry orifice. The view depicts an infusion access 803 and a valve 802 generally as previously described. The valve 802 in this instance is fitted over arrangement so that the valve subsists within and sealably blocks the infusion access 803 internal passageway 805. The fitted over arrangement includes a fitting 804 which serves to permanently retain the valve 802 at the infusion port 803 entry orifice.

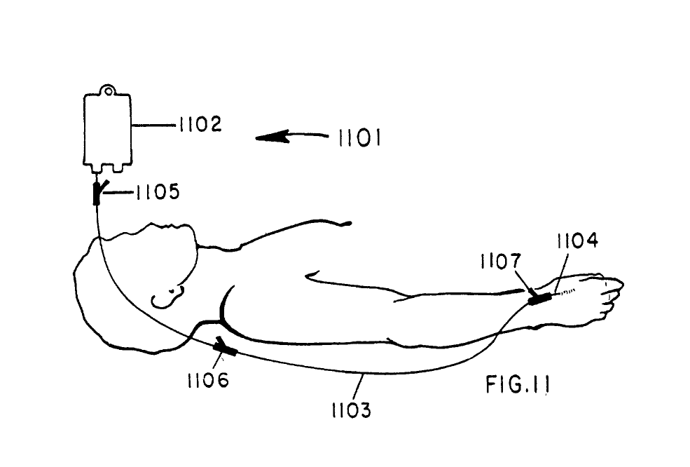

Referring to FIG. 9, a side view of an example needless and springless valve 901 is depicted. The valve 901 has a vertical dimension or height “a” and a horizontal dimension, width or diameter “b”. Both “a” and “b” can be varied to fit any particular desired infusion apparatus, and may be chosen based on the type of syringe tip to be accommodated.

FIG. 10 depicts a cross sectional side view 1001 of insertion of a non-sharp syringe tip 1002 into an example needleless and springless vascular infusion port apparatus 1003. The tip 1002 has pushed the pliable leaves or lips of the valve diaphragm out of the path of the tip to form an opening in the diaphragm through which the syringe tip can project for the purpose of infusing an infusate. The only necessary contact of the syringe to the infusion port apparatus 1003 is the syringe tip 1002 contacting the valve diaphragm.

The syringe need not include a luer lock or any other mechanism for retaining the syringe in juxtaposed position with respect to the infusion port, unless so desired. Upon removal of the tip 1002, the lips will resiliently return to their original position to re-engage and form and maintain a fluid-tight seal in the infusion port.

FIG. 11 depicts examples of locations on a vascular infusion assembly 1101 where a needless and springless vascular infusion port may be installed. The vascular infusion assembly 1101 may include a bottle or bag 1102 for containing an infusate to be infused into blood. A flexible tube 1103 connects the bag 1102 to an intravenous catheter 1104. One or more needleless vascular infusion port apparatuses 1 105, 1106 and 1107 may be placed on the vascular infusion assembly 1101 at appropriate intervals. For example, a needleless vascular infusion port apparatus 1105 might be placed 6 inches from the infusion bottle 1102. Or a needleless vascular infusion port apparatus 1106 might be placed 3 feet upstream from where an intravenous catheter is inserted into a vein (infusion site). Or a needleless vascular infusion port apparatus 1107 might be placed 6 inches upstream from where the intravenous catheter is inserted into a vein (infusion site). Other locations may be used as well.

The valve assembly may be made of any resilient, pliant material that can achieve a fluid-tight seal along its interface surfaces. The materials are desired to be pliant or pliable so that a syringe tip may be forceably cause leaves, lips or interface surfaces of a valve diaphragm to part to form opening for infusion of a desired infusate fluid. The material should be resilient so that the diaphragm returns to its original condition following syringe tip removal so that a fluid-tight seal is achieved and maintained. Such materials may include non-latex rubber, plastic, various polymer materials, and other materials such as deformable materials, elastic materials, elastomeric materials, and gels that are non-flowable at room temperature.

While the present devices have been described and illustrated in conjunction with a number of specific configurations, those skilled in the art will appreciate that variations and modifications may be made without departing from the principles herein illustrated, described, and claimed. The present invention, as defined by the appended claims, may be embodied in other specific forms without departing from its spirit or essential characteristics. The configurations of devices described herein are to be considered in all respects as only illustrative, and not restrictive. All changes which come within the meaning and range of equivalency of the claims are to be embraced within their scope.

We claim:

1. A needleless and springless vascular infusion port apparatus comprising:
a main infusion conduit body,
an internal passageway running through said main infusion conduit body,
an infusion access projecting said main infusion conduit body,
said infusion access being integrally formed with said main infusion conduit body,
an infusion access internal passageway,
said infusion access internal passageway being in fluid communication with said main infusion conduit body internal passageway,
an entry orifice on said infusion access,
a needleless valve assembly installed in said infusion access entry orifice,
said valve assembly having
a valve body, and
a valve closure,
said valve closure serving to interrupt said infusion access internal passageway to a fluid-tight seal therein,
said valve closure having a pliable and resilient openable diaphragm,
a plurality of leaves on said diaphragm,
a plurality of interface surfaces on said openable diaphragm,
said interface surfaces being capable of being parted by a non-sharp syringe tip to form an opening therebetween, said opening being formable by pressing a non-sharp syringe tip thereagainst, the opening being sufficient to permit a non-sharp syringe tip to protrude therethrough and directly infuse an infusate into the needless vascular infusion port;
wherein said valve diaphragm is sufficiently pliable to permit forceable insertion of a non-sharp syringe tip between said interfaces surfaces without damage to said diaphragm; and
wherein said valve diaphragm is sufficiently resilient to return to its original shape without assistance from a separate mechanical spring following removal of a syringe tip from an opening formed between said interface surfaces, and thereby re-create a fluid-tight seal in said infusion access internal passageway.

2. An apparatus as recited in claim 1 wherein at least a portion of said diaphragm is concave in shape when viewed from its distal surface.

3. An apparatus as recited in claim 2 wherein said concave shape tends to enhance resistance of said diaphragm to fluid backflow from the proximal side of said valve.

4. An apparatus as recited in claim 1 wherein said interface surfaces are formed by a slit in said diaphragm.

5. An apparatus as recited in claim 1 wherein said interface surfaces are formed by trifoliate leaves on said diaphragm.

6. An apparatus as recited in claim 1 wherein said interface surfaces are formed by a cross slit on said diaphragm.

7. An apparatus as recited in claim 1 further comprising a second openable valve diaphragm disposed within said infusion access internal passageway, said second openable valve diaphragm being situated in-line with said valve closure.

8. An apparatus as recited in claim 1 further comprising a staging area on the proximal side of said diaphragm.

9. An apparatus as recited in claim 1 further comprising constrained exit orifice located at the proximal end of said infusion access.

10. An apparatus as recited in claim 1 wherein said valve body is secured to said infusion access by a mechanism selected from the group consisting of a pressure fit, a friction fit, an adhesive, welding and mechanical fixation.

11. An apparatus as recited in claim 1 further comprising a rim of valve body material located about the exterior circumference of said infusion access entry orifice.

12. An apparatus as recited in claim 1 wherein said valve is located completely within said infusion access internal passageway.

13. An apparatus as recited in claim 1 further comprising a fitting located over said infusion access entry orifice that secures said valve in said infusion access internal passageway.

14. An apparatus as recited in claim 1 wherein said valve diaphragm includes a material selected from the group consisting of non-latex rubber, plastic, polymer materials, elastic materials, elastomeric materials, and gels which are non-flowable at room temperature.

15. A needleless and springless vascular infusion port apparatus comprising:
an infusion access,
an infusion access internal passageway,
an entry orifice on said infusion access,
a needleless valve assembly installed in said infusion access internal passageway,
said valve assembly having
a valve body, and
a pliable and resilient openable diaphragm,
a plurality of sealable lips on said diaphragm,
said diaphragm serving to interrupt said infusion access internal passageway to form a fluid-tight seal therein,
said lips being capable of being parted to form an opening therebetween in response to pressure placed thereagainst by a non-sharp syringe tip, the opening being sufficient to permit a syringe tip absent a sharp to protrude therethrough and directly infuse an infusate into the needless vascular infusion port;
wherein said valve diaphragm is sufficiently pliable to permit forceable insertion of a syringe tip absent a sharp between said lips without damage to said valve diaphragm; and
wherein said valve diaphragm is sufficiently resilient to return to its original shape without assistance from a separate mechanical spring following removal of a syringe tip from an opening formed between said lips, and thereby re-create a fluid-tight seal in said infusion access internal passageway.

16. An apparatus as recited in claim 15 wherein at least a portion of said diaphragm is concave in shape when viewed from its distal surface.

17. An apparatus as recited in claim 16 wherein said concave shape tends,to enhance resistance of said diaphragm to fluid backflow from the proximal side of said valve.

18. An apparatus as recited in claim 15 wherein said lips are formed by trifoliate leaves on said diaphragm.

19. An apparatus as recited in claim 15 wherein said lips are formed by cross slits on said diaphragm.

20. An apparatus as recited in claim 15 further comprising a second openable valve diaphragm disposed within said infusion access internal passageway.

21. An apparatus as recited in claim 15 wherein said valve body is secured to said infusion access by a mechanism selected from the group consisting of a pressure fit, a friction fit, an adhesive, welding and mechanical fixation.

22. An apparatus as recited in claim 15 further comprising a rim of valve body material located about the exterior circumference of said infusion access entry orifice.

23. An apparatus as recited in claim 16 wherein said valve is located completely within said infusion access internal passageway.

24. An apparatus as recited in claim 15 further comprising a fitting located over said infusion access entry orifice that secures said valve in said infusion access internal passageway.

25. An apparatus as recited in claim 15 wherein said valve diaphragm includes a material selected from the group consisting of non-latex rubber, plastic, polymer materials, elastic materials, elastomeric materials, and gels which are non-flowable at room temperature.

26. A needleless and springless vascular infusion port apparatus comprising:
an infusion access,
an infusion access internal passageway,
an entry orifice on said infusion access,
a needleless valve assembly installed in said infusion access internal passageway,
said valve assembly having
a valve body, and
a resilient openable diaphragm,
a plurality of pliable, resilient leaves on said diaphragm,
a plurality of sealable lips on said leaves,
said diaphragm serving to interrupt said infusion access internal passageway to form a fluid-tight seal therein,
said lips being capable of being parted to form an opening therebetween, the opening being sufficiently pliable to permit a syringe tip absent a sharp to protrude therethrough and directly infuse an infusate into the needless vascular infusion port;
wherein said valve diaphragm is sufficiently pliable to permit forceable insertion of a syringe tip absent a sharp between said lips without causing damage to said diaphragm; and
wherein said valve diaphragm is sufficiently resilient to return to its original shape following removal of a syringe tip from an opening formed between said lips, and thereby employ said lips to sealably re-create a fluid-tight seal in said infusion access internal passageway;
wherein at least a portion of said diaphragm is concave in shape when viewed from its distal surface; and
wherein said valve diaphragm includes a material selected from the group consisting of non-latex rubber, plastic, polymer materials, elastic materials, elastomeric materials, and gels that are non-flowable at room temperature.
